(19) United States
(12) Patent Application Publication
(10) Pub. No.: US 2017/0007845 A1
(43) Pub. Date: Jan. 12, 2017

(54) Method and Apparatus for Proximity Control in Cold Plasma Medical Devices

(71) Applicant: Plasmology4, Inc., Scottsdale, AZ (US)

(72) Inventors: Marc C. JACOFSKY, Phoenix, AZ (US); Michel H. YOON, Alameda, CA (US)

(73) Assignee: Plasmology4, Inc., Scottsdale, AZ (US)

(21) Appl. No.: 15/262,790

(22) Filed: Sep. 12, 2016

Related U.S. Application Data
(62) Division of application No. 14/142,333, filed on Dec. 27, 2013.
(60) Provisional application No. 61/747,104, filed on Dec. 28, 2012.

(51) Int. Cl. A61N 5/06; A61N 1/44; A61B 90/00

(52) U.S. Cl. CPC A61N 5/0616; A61B 90/06; A61N 1/44; A61B 2090/061; A61N 2005/0643; A61N 2005/0662; A61N 2005/0663; A61N 2005/067

(57) ABSTRACT

Methods and apparatus are described that use an array of light sources that project converging light beams to control treatment distance. This approach controls treatment distance without contacting the patient or increasing the risk of pathogenic contamination. The approach can be used to control an optimal distance, and is compatible with various medical treatment devices including cold plasma treatment devices.

### CROSS REFERENCE TO RELATED APPLICATIONS

This application is a divisional of U.S. application Ser. No. 14/142,333, filed Dec. 27, 2013, which claims the benefit of U.S. Provisional Application No. 61/747,104, filed Dec. 28, 2012 and entitled “Method and Apparatus for Proximity Control in Cold Plasma Medical Devices,” which is incorporated herein by reference in its entirety.

This application is related to U.S. Provisional Application No. 60/913,369, filed Apr. 23, 2007; U.S. patent application Ser. No. 12/038,159, filed Feb. 27, 2008 (which issued as U.S. Pat. No. 7,633,231); and U.S. patent application Ser. No. 13/620,236, filed Sep. 14, 2012, each of which are herein incorporated by reference in their entireties.

### BACKGROUND

Field of the Art

The present invention relates to devices and methods for cold plasma generation, and, more particularly, to such devices and methods that control the proximity distance of a cold plasma device to a treatment area.

Background Art

Cold plasma medicine is a relatively new and growing field of medicine. Most cold plasma medical applications focus on disease eradication including; bacteria, viruses, cancers, and dermatological disorders. There exist multiple methodologies to produce cold plasmas for medicine including dielectric barrier discharge through atmospheric air and gas plasma torches. Gas plasma torches may be further subdivided into equilibrium and non-equilibrium plasmas depending upon the supplied power and electrode configuration. Equilibrium plasmas generally have a higher electron density, but operate at higher temperatures. All of the existing plasma generation methods may be used with a variety of feed gasses from atmospheric air to pure noble gasses or mixtures thereof (He, Ar, N, and O for example).

Regardless of the method used to generate a therapeutic cold plasma, the distance that the plasma source is held from the treatment target is very important to ensure both the safety and efficacy of the treatment. In an equilibrium argon plasma, safety issues arise as the temperature varies dramatically within the plasma stream and can lead to burns if held too close to the skin. In cold plasma devices, the colder temperature of the plasma does not pose a safety issue, but the distance poses an efficacy of treatment issue. For example, in floating electrode dielectric barrier discharge devices, if the distance is too great, no plasma is ignited due to the dielectric properties of air and the perceived absence of the required second grounded electrode (target surface).

### BRIEF SUMMARY OF THE INVENTION

An embodiment is described of a cold plasma device having two or more visible beams of light that converge at a predetermined target distance associated with the treatment protocol when using the cold plasma device.

A further embodiment is described of a method of producing cold plasma. The method includes applying cold plasma from a cold plasma device to a treatment area having a predetermined target distance associated with a treatment protocol. The method further includes emitting two or more visible beams of light that converge at the predetermined target distance.

### BRIEF DESCRIPTION OF THE DRAWINGS/FIGURES

FIG. 1 illustrates a ring adapter used to control treatment distance.

FIG. 2 illustrates a floating electrode DBD device utilizing a Z-Micro positioner.

FIG. 3 illustrates a projection embodiment of proximity control device for a cold plasma treatment device, in accordance with an embodiment of the present disclosure.

FIG. 4 illustrates a cone-shaped shroud attached to a hand-held cold plasma device, in accordance with an embodiment of the present disclosure.

FIG. 5 illustrates a front view of a cold plasma device with the array of diodes installed below the attachment point for the tips, in accordance with an embodiment of the present disclosure.

FIG. 6 illustrates a top view of a cold plasma device (e.g., a multi-frequency harmonic-rich cold plasma (MFHCP) device using the '369 patent family) highlighting the different converging light paths for various treatment spacing depending upon the protocol in use with each particular combination of cold plasma tip and gas composition, in accordance with an embodiment of the present disclosure.

FIGS. 7A and 7B illustrate a proximity ring illumination approach, in accordance with an embodiment of the present disclosure.

FIGS. 8A, 8B and 8C illustrate a cold plasma applicator having a disposable tip that includes a built-in prismatic surface, in accordance with an embodiment of the present disclosure.

FIGS. 9A, 9B and 9C illustrate a cold plasma applicator that includes a non-disposable built-in prismatic surface, in accordance with an embodiment of the present disclosure.

FIGS. 10A, 10B and 10C illustrate a cold plasma applicator that includes a light pipe, in accordance with an embodiment of the present disclosure.

FIGS. 11A, 11B and 11C illustrate a cold plasma applicator that includes a light pipe, in accordance with an embodiment of the present disclosure.

FIG. 12 illustrates a flowchart of a method that provides treatment distance control of a cold plasma device, in accordance with an embodiment of the present disclosure.

### DETAILED DESCRIPTION OF THE INVENTION

Cold temperature plasmas have attracted a great deal of enthusiasm and interest by virtue of their provision of plasmas at relatively low operating gas temperatures. This provision is of interest to a variety of applications, including wound healing, anti-bacterial processes, various other medical therapies and sterilization.

As previously mentioned, maintaining an optimal distance from the treatment surface is very important for both the safety and the effectiveness of the plasma. However, a proximity device that comes into direct contact with a wound bed can pose a risk of introducing pathogens onto that surface. Furthermore, different regulations and standards may apply to a device that contacts a patient versus one that does not. For these reasons it is desirable to have a non-contact means to regulate the distance that a plasma medicine device is held from the treatment target.

At present, there are several mechanisms in use when a specific distance is required for various methods of medical treatment. For instance, in ultrasonography an adjustable stand-off is used. The stand-off maintains the distance between the transducer and skin in order to bring the area of investigation into the focal zone.

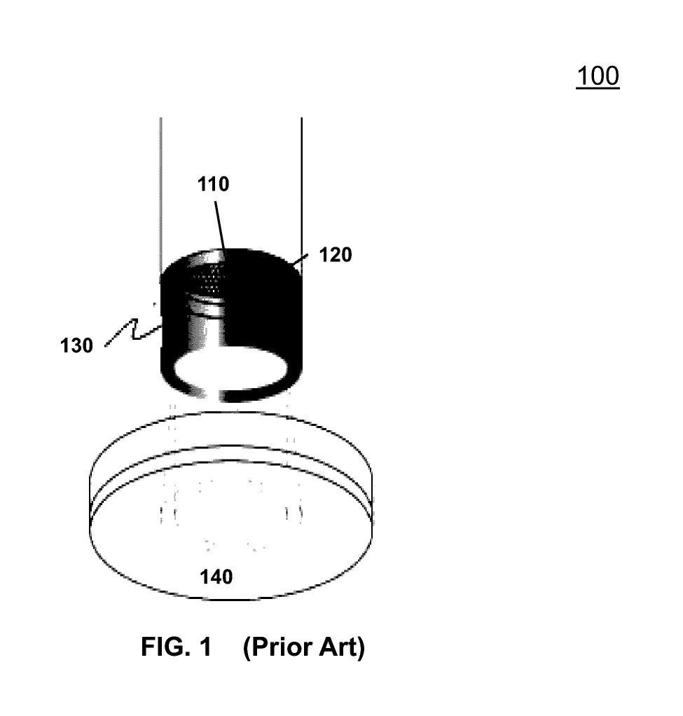

In at least one current cold plasma application, ring adapters of various heights are employed when treating bacterial cultures in vitro or animal wounds in vivo. FIG. 1 (adapted from Li et al., “Optimizing the Distance for Bacterial Treatment Using Surface Micro-discharge Plasma,” February 2012) shows the use of ring adapter 130 with electrode 120 from plasma device 110 during a plasma treatment of agar sample 140. These ring shaped stand-offs are used to control the distance between the treatment device and the treatment target. The ring adapters touch both the target surface and the distal surface of the plasma delivery device. These rings are disadvantageous because they represent an additional step in the treatment process, can be painful to insert in and remove from the wound bed, and are a potential source of infection transmission.

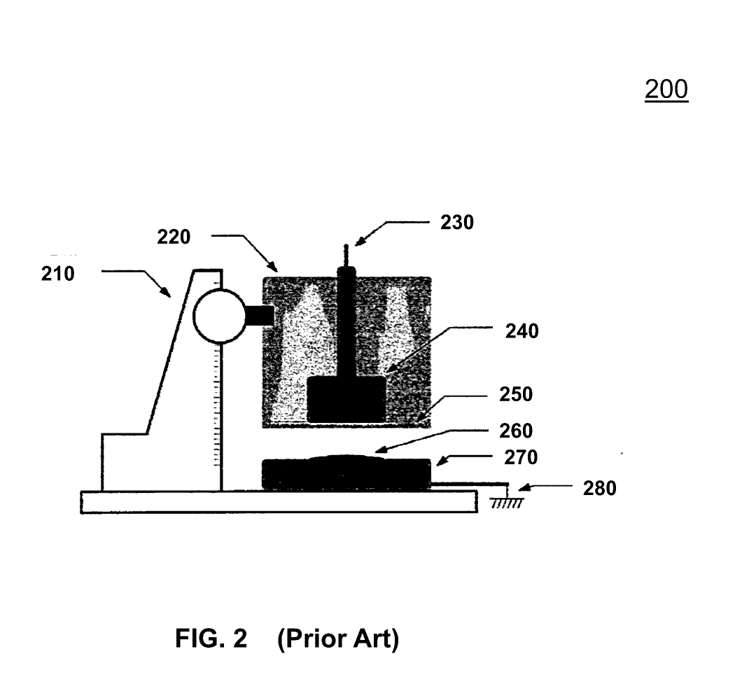

FIG. 2 (adapted from Fridman et al., “Use of Non-Thermal Atmospheric Pressure Plasma Discharge for Coagulation and Sterilization of Surface Wounds,” 2005) shows the use of a positioner 210 for positioning DBD device (high voltage port 230, teflon coating 220, copper electrode 240 inside quartz dielectric 250) for application to blood sample 260 in holder 270 that is in contact with ground 280. This adjustable stand has been utilized to control distances between a dielectric barrier discharge (DBD) plasma device and the intended treatment target, but this is of limited practical value to a physician treating a live patient, both of whom could and will be moving, during treatment time. Such a system does not allow for rapid micro-adjustments to be made, real time, at the discretion of the medical professional as would be required in actual situations. Furthermore, this adjustable stand does not alleviate the threat of infection resulting from the contact between the fixture and the treatment target.

In summary, existing approaches for cold plasma DBD devices to maintain an optimal distance pose significant risks of pathogen introduction when the proximity device comes into direct contact with a wound bed can pose a risk of introducing pathogens onto that surface. Thus, it is desirable to have a non-contact means to control the distance that a plasma treatment device is held from the treatment target. It is further desirable to devise approaches to control treatment distance for cold plasma jet devices.

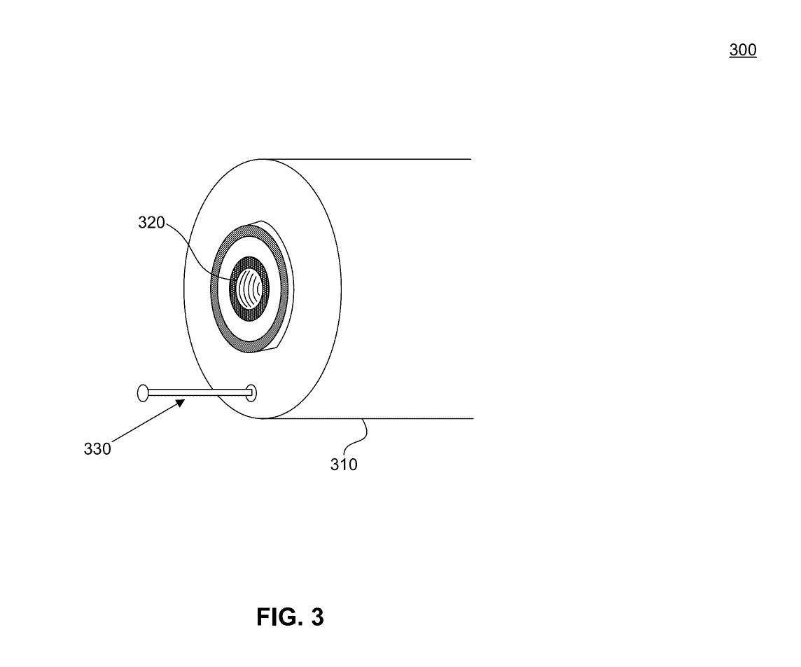

FIG. 3 illustrates one approach to control treatment distance in a cold plasma jet device. Cold plasma device 310 provides a cold plasma from aperture 320 for treatment purposes. Cold plasma device 310 introduces a small, non-conductive, projection 330 with a flat smooth face to the functioning end of cold plasma device 310. This projection could be provided in variable lengths depending on the type of treatment being performed and could be disposable to minimize infection risk. This approach would limit the minimum distance of treatment but would not limit the maximum treatment distance effectively.

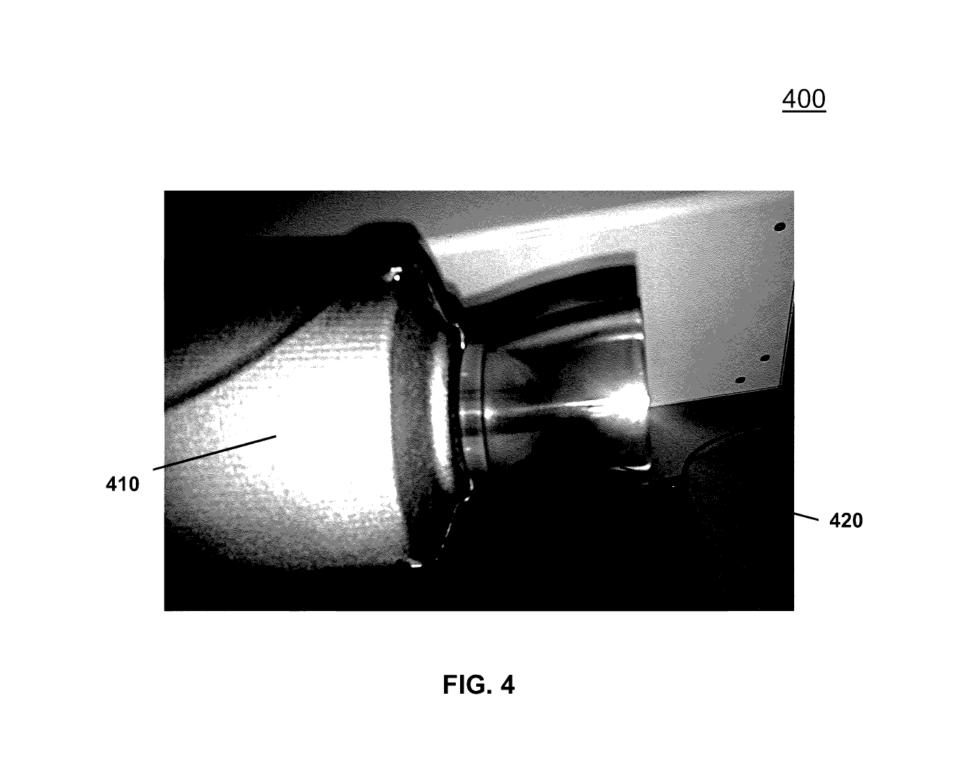

FIG. 4 illustrates a cone-shaped shroud 420 attached to a hand-held cold plasma device 410. This integrated shroud embodiment can be used, in part, to accurately control the treatment distance in a cold plasma device. Again, this limits a minimum treatment distance but does not control for maximum distances. Maximum distance would be limited by instructing the user to keep the projection “as close as possible to the treatment site without contact.” However, contact with the treatment site is still likely to occur with patient and operator movement, which could cause discomfort, lead to contamination, and open additional regulatory challenges. Thus, what is clearly needed is a non-contact means of indicating treatment distances that are too close or too far from the optimal intended distance.

The confounding factor with all of the aforementioned devices is that they require, or likely lead to, mechanical contact with the patient undergoing treatment. The challenge is to create a device that will guide the optimum treatment distance between the patient and treatment device being utilized by the medical professional, both of whom are constantly in motion, without creating physical contact between the device and the patient's body. A method is needed that controls the treatment distance without contacting the patient and increasing the risk of pathogen transfer.

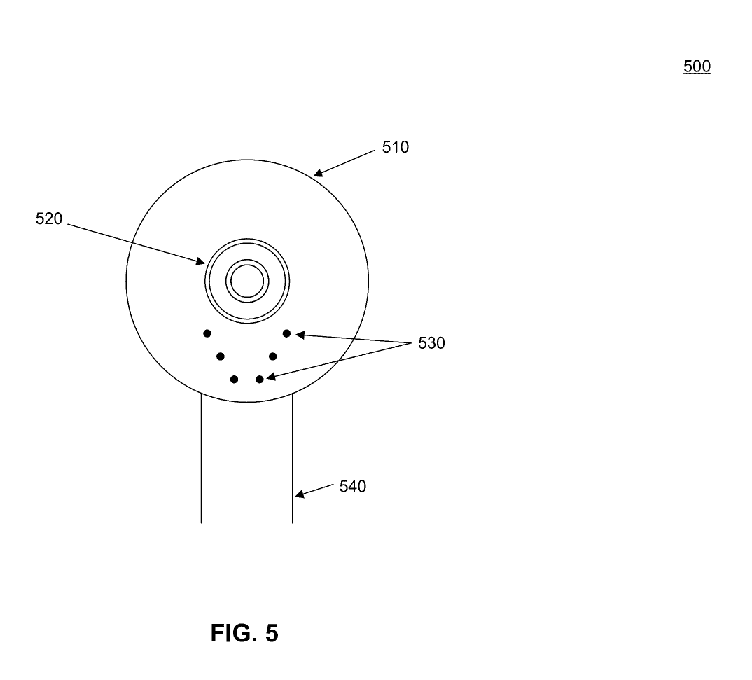

In many therapeutic situations, 2.5 cm appears to be the optimal treatment distance when using a cold plasma device (e.g., such as the multi-frequency harmonic-rich cold plasma (MFHCP) generation units described in U.S. Provisional Patent Application No. 60/913,369, filed Apr. 23, 2007; U.S. Non-provisional application Ser. No. 12/038,159, filed Feb. 27, 2008 (that has issued as U.S. Pat. No. 7,633,231) and the subsequent continuation applications (collectively “the '369 patent family”), which are incorporated herein by reference), though the effective range varies from <1- 22 3 cm. It is emphasized that the term “cold plasma device,” when used herein, refers to any cold plasma device irrespective of how the cold plasma is generated. In particular, the term “cold plasma device” is not limited to an MFHCP cold plasma device. The MFHCP cold plasma device is an example of a cold plasma device. Cold plasma devices may also be used with a tip, as for example described in U.S. Non-provisional application Ser. No. 13/620,236 (“the '236 application”), filed Sep. 14, 2012, which is incorporated herein by reference. An embodiment of the present disclosure envisions a cold plasma device (e.g., a cold plasma device as described in the '369 patent family) that contains an array of light sources (e.g., light emitting diodes, laser diodes, etc.) on the front of the device that project converging light beams (as illustrated in FIG. 5). In the simplest embodiment, a single pair of converging LEDs is placed adjacent to the plasma-emitting orifice at the terminal end of the plasma applicator. The angle of convergence is set such that at the optimum treatment distance (e.g., 2.5 cm) the two light beams fowl a single dot on the treatment surface. At distances closer and further than the optimal distance, two distinct lights appear on the treatment target. In another embodiment, a plurality of convergent light sources is provided and the desired treatment dictates which set of light sources in the array are activated. For example, if a particular treatment tip (e.g., a tip as described in the '236 application), gas composition, or other system setting is selected, the light source corresponding to the optimal treatment distance for the current device settings are automatically invoked. The plurality of light sources can indicate different ideal treatment distances by either selecting lights with the same spacing but different angles of convergence, or the same angle of convergence with different spacing distance on the applicator, the equivalence of which should be apparent to one skilled in the art. A servo-mechanical system could also be used to vary the angle of convergence between a single set of light sources rather than requiring a plurality. A second embodiment could contain an adjustable, or removable, lens in front of each light source to obtain the same effect, a varying target zone to optimize plasma treatment, but with fewer diodes.

As noted above, FIG. 5 illustrates a front view 500 of a cold plasma device 510 (e.g., a multi-frequency harmonic-rich cold plasma (MFHCP) device described in the '369 patent family) with the array 530 of light sources (e.g., LED sources, laser diode sources) installed below the attachment point 520 (e.g., attachment ring) for the attached tips. Tips attach, either permanently or in a disposable fashion, to cold plasma device 510 and provide an aperture through which cold plasma emanates from cold plasma device 510. Tips are configured to provide cold plasma commensurate with different treatment protocols. Thus, tips come in different sizes and incorporate different materials within the tips to configure the cold plasma appropriate to different treatment protocols. Optional handle 540 is shown in front view 500 of cold plasma device 510.

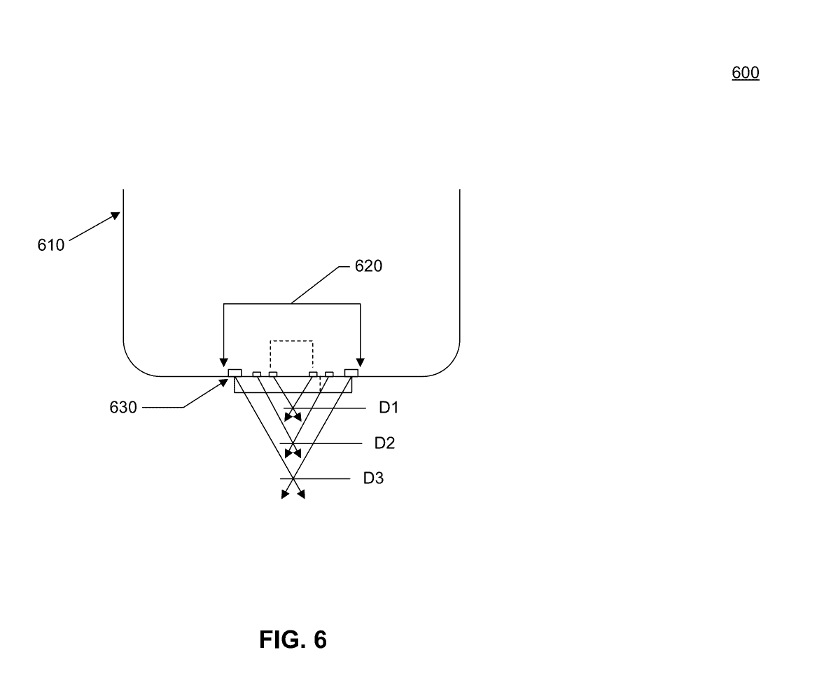

Returning to array 530 of light sources, the placement of array 530 on cold plasma device 510 avoids interference between the proximity-control light beams and the plasma stream. Each pair of diodes could corresponds to a unique set of tips and gas composition combinations for various treatment protocols; thus, allowing the cold plasma treatment device to be fully capable of achieving multiple convergent zones (e.g., as illustrated in FIG. 6) at different angles.

FIG. 6 illustrates a side view of the body 610 of a cold plasma device (e.g., a MFHCP device, as described in the '369 patent family) highlighting the different converging light paths for various treatment spacing depending upon the protocol in use with each particular combination of cold plasma tip and gas composition. Diode array 620 includes a number of pairs of light sources 630 (e.g., LED sources, laser diode sources). The beams would be designed to converge at predetermined target distances (e.g., D1, D2, D3), within acceptable tolerances. In an exemplary embodiment, D1, D2 and D3 may be 1-2 cm, 2-3 cm and 3-4 cm respectively.

The diodes used in the cold plasma device could be standard light emitting diodes (“LED”), laser diodes, or any other mono or polychromatic light source known to those skilled in the art, as long as they produce a visible beam of light that can be seen to converge at the target distances. In addition to the benefits of keeping the plasma stream optimized as to the proximity to the treatment area, the light source itself could be designed to have an additional benefit toward established would healing protocols.

In 1998, NASA embarked on Phase I of a series of studies to determine the effectiveness of LED's irradiation in wound healing. In vitro experiments demonstrably showed cell growth of 140-200% in both mouse and rat derived fibroblasts, and in rat derived skeletal muscle. Increase in growth of 155-171% of normal human epithelial cells was observed in vitro. Wound size decreases of up to 36% in conjunction with hyperbaric oxygen were observed in ischemic rat models. Improvement of greater than 40% in musculoskeletal training injuries was observed in Navy SEAL team members. Decreased wound healing time by 50% was observed by selected Navy crewmen (H. T. Whelan et al., 2001). The diodes being used in the cold plasma device to accurately gage the target zone of optimized treatment could be of similar power and wavelength (wavelengths between 500 and 1000 nm, or more specifically wavelengths of 670, 720, and 880 nm, at power levels between 40 mW/cm² and 55 mW/cm²) to those used in the studies above, or used in conjunction with the visible-beam diodes, to enhance wound treatment, thereby presenting a combination plasma and light therapy device.

In another embodiment, each LED of the converging pair may be of different colors. For example, a yellow LED on one side and a blue LED on the other. In this embodiment a green light is produced when the light sources converge on a single point. Additionally, this provides a means for the user to easily determine if the treatment distance is too close or too far when the LEDs are not in alignment. With a pair of the same color, the applicator must be moved in and out to determine if the applicator is too close or too far. With different colored LEDs it is readily apparent if the light has crossed to the other side (too far) or remains on the same side of the applicator (too close).

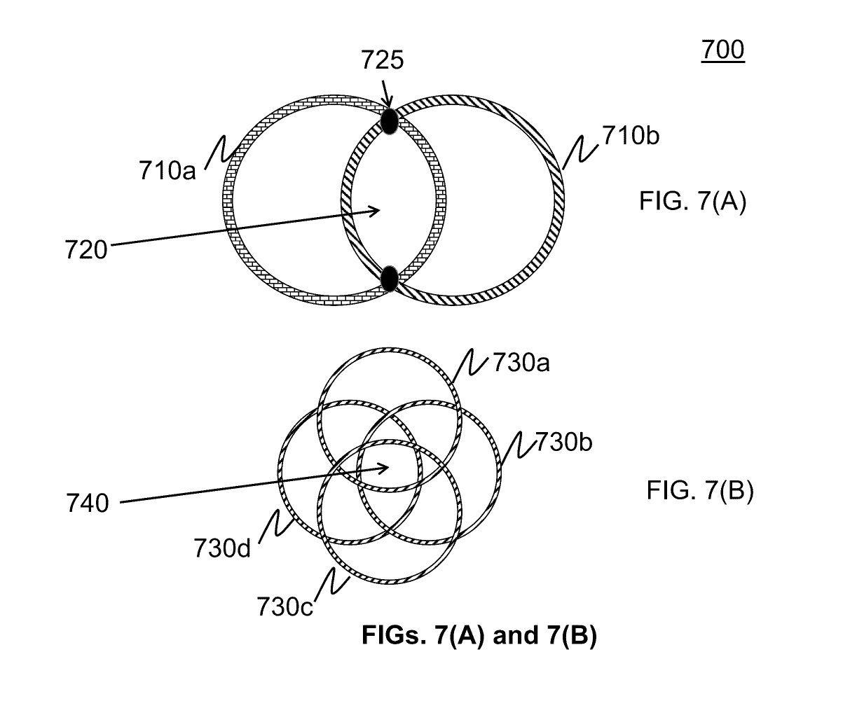

In a further embodiment of the present disclosure, FIG. 7A illustrates a proximity ring illumination pattern 700 configured to provide proximity guidance with a cold plasma applicator. Two light sources (i.e., a pair of light sources) are coupled to the distal end of a cold plasma applicator, with the resulting circular light projections 710a, 710b from the two light sources converging at the optimal operating distance, Area 720, which is common to circular light projections 710a, 710b, represents the optimal treatment zone of the cold plasma applicator. In a still further embodiment, circular light projections 710a, 710b may be different colors. The different colored lights would combine to form a third color where they intersect to thereby add a further visual cue that the correct operating distance has been reached. For example, circular light projections 710a, 710b may be blue and yellow respectively, with a resulting green for the third color,

Furthermore, the pair of lights sources can be configured to support different operating distances associated with different cold plasma treatment protocols. In a further embodiment illustrated in FIG. 7B, multiple pairs of light sources may be coupled to the distal end of a cold plasma applicator, with the resulting circular light projections 730a, 730b, 730c, 730d, . . . from the multiple pairs of light sources converging at the optimal operating distance. Area 740, which is common to all circular light projections 730a, 730b, 730c, 730d, . . . represents the optimal treatment zone of the cold plasma applicator.

Proximity ring illumination pattern 700 embodiments offer a number of advantages. First, these embodiments enable both the proximity (i.e., proper operating distance) and treatment area to be defined for optimization of various cold plasma treatment protocols. Second, the surface topography of the treatment area has a lesser effect on the converging circular light projections compared with solid light source “dots.” In all of the proximity ring illumination pattern 700 embodiments, the light sources may be any suitable light source, including LED sources and laser diode sources.

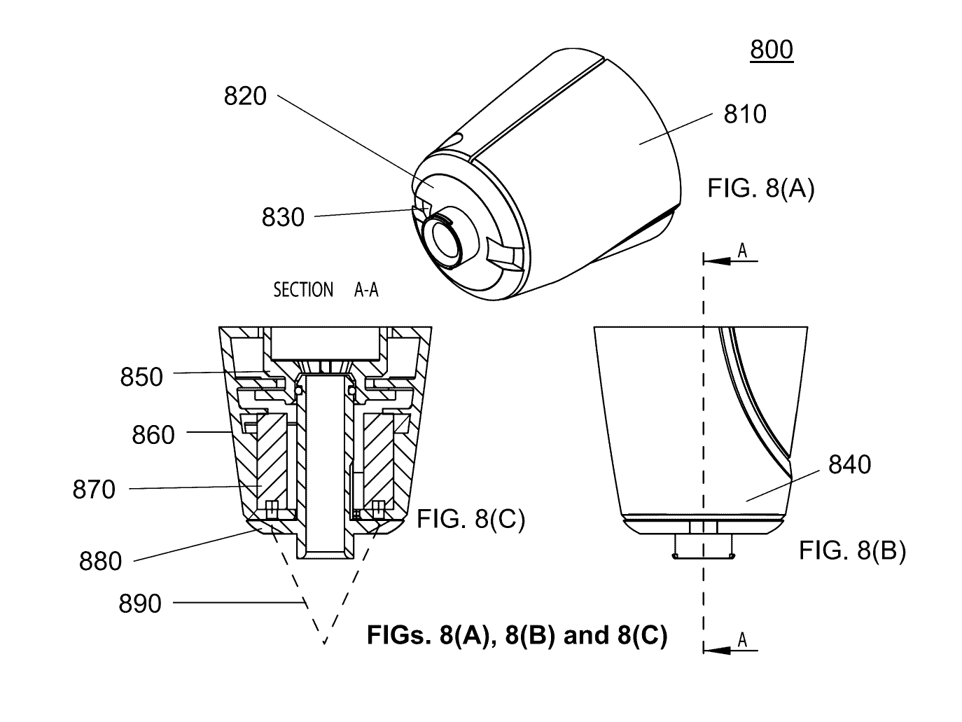

FIGS. 8-11 illustrate additional embodiments of the present disclosure. FIGS. 8A-8C illustrate a cold plasma applicator 810 having a disposable tip 820 that includes a built-in prismatic surface 830. FIG. 8B provides a plan view 840 of cold plasma applicator 810, whose cross-sectional view A-A is shown in FIG. 8C. FIG. 8C shows a portion of cold plasma generation module 850 inside cold plasma applicator housing 860. Light source(s) 870 (e.g., laser diode, LED) emit light that is directed to prism 880 (e.g., lens) that emerge as light beams 890. Prism 880 is configured to provide the appropriate light beam paths consistent with the desired convergence point associated with the tip and its corresponding treatment protocol. Placement of the light sources is based on optical path considerations, as well as the need to ensure that the light sources do not inadvertently provide a false ground for the cold plasma.

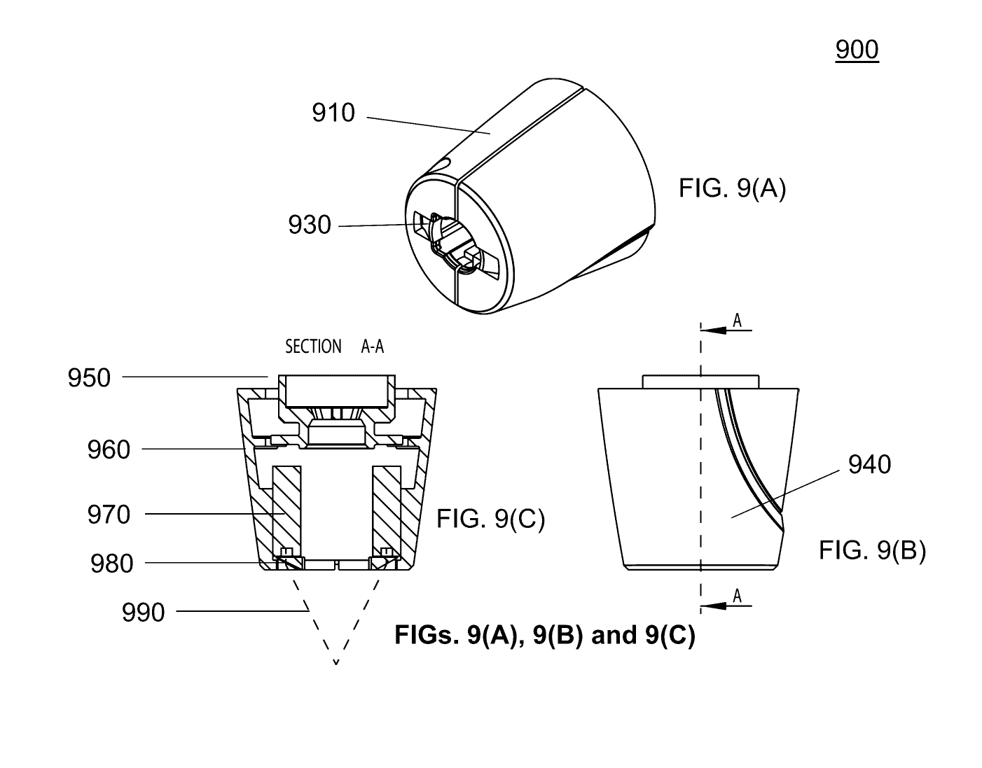

FIGS. 9A-9C illustrate a cold plasma applicator 910 that includes a non-disposable built-in prismatic surface 930. FIG. 9B provides a plan view 940 of cold plasma applicator 910, whose cross-sectional view A-A is shown in FIG. 9C. FIG. 9C shows a portion of cold plasma generation module 950 inside cold plasma applicator housing 960. Light source(s) 970 (e.g., laser diode, LED) emit light that is directed to prism 980 (e.g., lens) that emerge as light beams 990. Prism 980 is configured to provide the appropriate light beam paths consistent with the desired convergence point associated with the tip and its corresponding treatment protocol. Placement of the light sources is based on optical path considerations, as well as the need to ensure that the light sources do not inadvertently provide a false ground for the cold plasma.

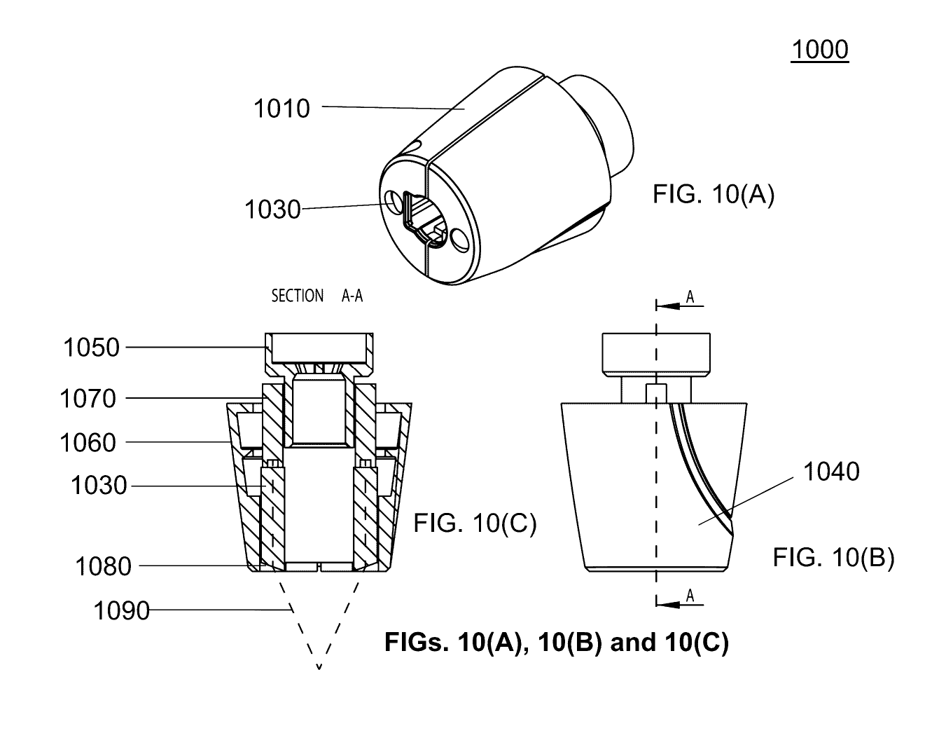

FIGS. 10A-10C illustrate a cold plasma applicator 1010 that includes a light pipe 1030. FIG. 10B provides a plan view 1040 of cold plasma applicator 1010, whose cross-sectional view A-A is shown in FIG. 10C. FIG. 10C shows a portion of cold plasma generation module 1050 inside cold plasma applicator housing 1060. Light source(s) 1070 (e.g., laser diode, LED) emit light that is directed along light pipe 1030 to prism 1080 (e.g., lens) that emerge as light beams 1090. Prism 1080 is configured to provide the appropriate light beam paths consistent with the desired convergence point associated with the tip and its corresponding treatment protocol. Placement of the light sources 1070 and length of light pipe 1030 is based on optical path considerations, as well as the need to ensure that the light sources do not inadvertently provide a false ground for the cold plasma.

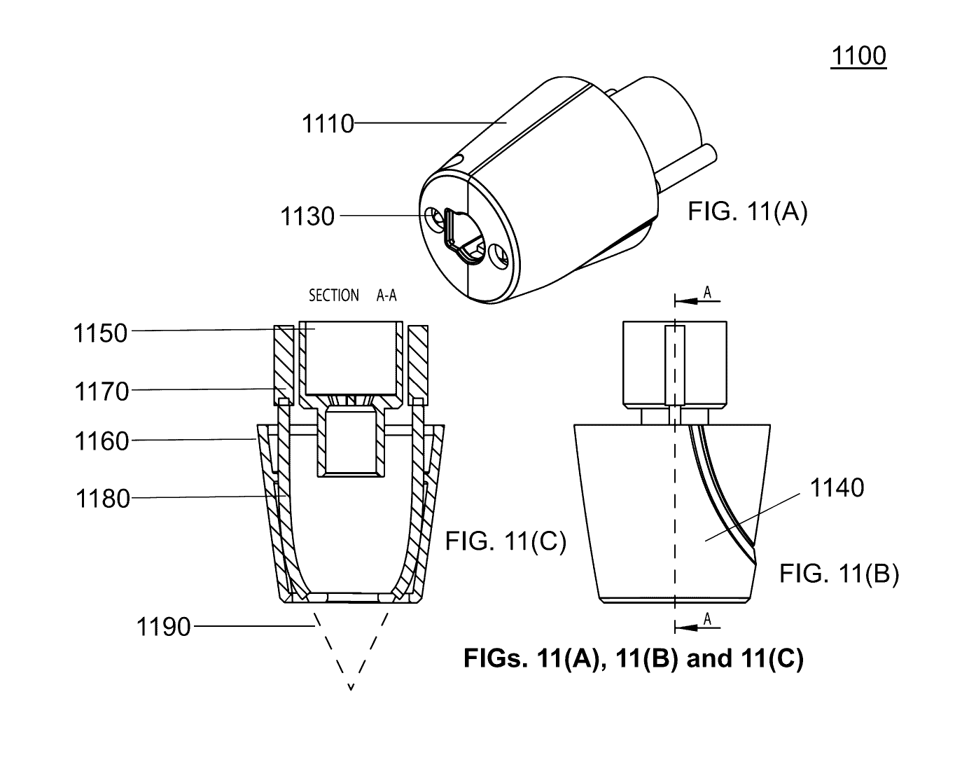

FIGS. 11A-11C illustrate a cold plasma applicator 1110 that includes a light pipe 1130. FIG. 11B provides a plan view 1140 of cold plasma applicator 1110, whose cross-sectional view A-A is shown in FIG. 11C. FIG. 11C shows a portion of cold plasma generation module 1150 inside cold plasma applicator housing 1160. Light source(s) 1170 (e.g., laser diode, LED) emit light that is directed along fiber optics cable 1180 that emerge as light beams 1190. Fiber optics cable 1180 is configured to provide the appropriate light beam paths consistent with the desired convergence point associated with the tip and its corresponding treatment protocol. Placement of the light sources 1170 and length of fiber optics cable 1180 is based on optical path considerations, as well as the need to ensure that the light sources do not inadvertently provide a false ground for the cold plasma. Fiber optics cable 1180 can be physically configured to direct light beam paths in the desired directions, or associated with prisms (not shown in FIGS. 11A-11C) that can be placed at the terminus of fiber optics cable 1180.

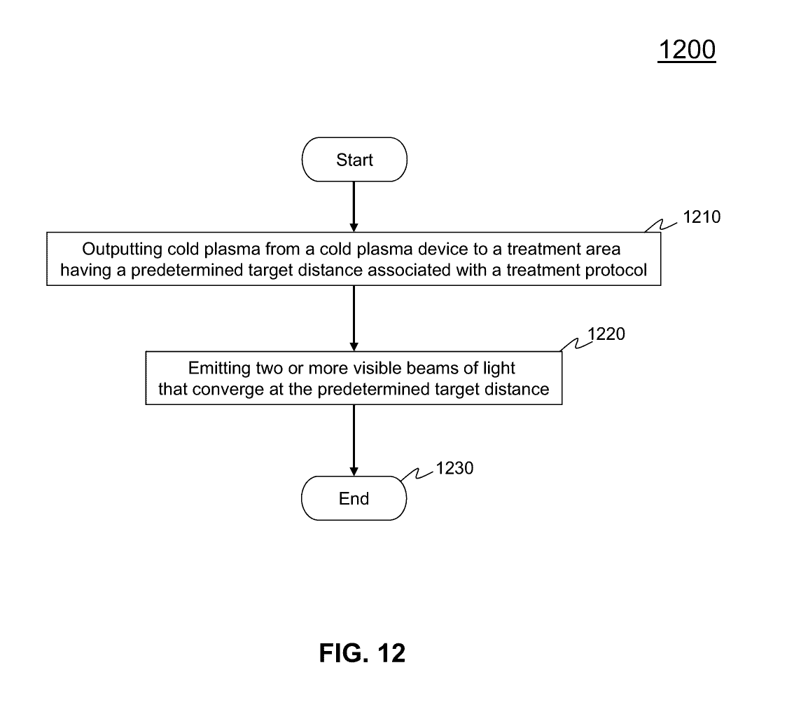

FIG. 12 provides a flowchart of a method that provides treatment distance control of a cold plasma device, according to an embodiment of the current invention.

The process begins at step 1210. In step 1210, cold plasma is output from a cold plasma device to a treatment area having a predetermined target distance associated with a treatment protocol. In an embodiment, cold plasma device 510 provides the cold plasma to be applied to the treatment area in accordance with a treatment protocol.

In step 1220, two or more visible beams of light are emitted that converge at the predetermined target distance. In an embodiment, light source array 620 provides visible beams of light that converge at distances D1, D2 and D3, as illustrated in FIG. 6.

At step 1230, method 1200 ends.

Although the above description has used the '369 patent family as the baseline cold plasma device, the scope of the present invention is not limited to the '369 patent family baseline. The '369 patent family baseline is merely exemplary and not limiting, and therefore embodiments of the present invention include the deployment of the above proximity features to cold plasma generation devices in general.

It is to be appreciated that the Detailed Description section, and not the Summary and Abstract sections, is intended to be used to interpret the claims. The Summary and Abstract sections may set forth one or more, but not all, exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A method comprising:
selecting a treatment protocol;
selecting a tip that corresponds with the selected treatment protocol, wherein the selected tip is configured to emit two or more visible beams of light that converge at a predetermined distance from the selected tip, the predetermined distance being associated with the selected treatment protocol;
attaching the selected tip to a cold plasma device;
positioning the cold plasma device relative to a treatment area so that the predetermined distance coincides with the treatment area; and
outputting cold plasma from the cold plasma device to the treatment area.

2. The method of claim 1, further comprising:
using an appearance of the two or more visible beams converging at the predetermined distance to continuously maintain a treatment distance associated with the selected treatment protocol throughout application of the cold plasma to the treatment area

3. The method of claim 1, further comprising:
using a gas having a predetermined gas composition, wherein the predetermined gas composition is compatible with the selected treatment protocol, the selected tip and the predetermined distance.

4. The method of claim 1, wherein a wavelength of the two or more visible beams of light is compatible with the treatment protocol.

5. The method of claim 1, wherein an intensity of the two or more visible beams of light is compatible with the treatment protocol.

6. The method of claim 1, wherein light from the two or more visible beams of light forms a part of the treatment protocol.

7. The method of claim 1, wherein the two or more visible beams of light comprise different colors that converge at the predetermined target distance to provide a third color, the third color being a visual cue of a correct operating distance from the selected tip to the treatment area.

8. The method of claim 1, wherein the two or more visible beams of light are generated by light emitting diode devices or laser diode devices.

9. The method of claim 1, wherein outputting the cold plasma includes outputting via the selected tip coupled to the cold plasma device, the selected tip having an aperture for output of the cold plasma, and wherein emitting the two or more visible beams of light includes using two or more lenses integrated within the selected tip to direct the visible beams of light to converge at the predetermined target distance.

10. The method of claim 1, wherein emitting two or more visible beams of light includes directing the two or more visible beams of light to intersect to delineate a treatment zone defined by an enclosed area formed by the intersection of the two or more visible beams of light.

11. The method of claim 1, wherein the treatment area at the predetermined target distance is remote from the cold plasma device.

12. The method of claim 1, wherein the two or more visible beams of light are generated by light emitting diode devices or laser diode devices.

13. An apparatus comprising:
a tip that corresponds to a selected treatment protocol, wherein the tip is configured to emit two or more visible beams of light that converge at a predetermined distance from the tip, the predetermined distance being determined by the selected treatment protocol;
a cold plasma device configured to produce cold plasma, wherein the tip is attached to the cold plasma device, wherein the predetermined distance coincides with a treatment area, and wherein the produced cold plasma from the cold plasma device is provided to the treatment area.

14. The apparatus of claim 1, wherein the predetermined target distance is determined based on one or more of a tip attached to the cold plasma device and a predetermined gas composition.

15. The apparatus of claim 1, wherein a wavelength of the two or more visible beams of light is compatible with the treatment protocol.

16. The apparatus of claim 1, wherein an intensity of the two or more visible beams of light is compatible with the treatment protocol.

17. The apparatus of claim 1, wherein light from the two or more visible beams of light forms a part of the treatment protocol.

18. The apparatus of claim 1, wherein the two or more visible beams of light comprise different colors that converge at the predetermined target distance to provide a third color, the third color being a visual cue of a correct operating distance.

19. The apparatus of claim 1, wherein the two or more visible beams of light are generated by light emitting diode devices or laser diode devices.

20. The apparatus of claim 1, wherein the tip includes an aperture and two or more lenses integrated within the tip, wherein the aperture allows for output of the cold plasma, and where the lenses are configured to direct the visible beams of light to converge at the predetermined target distance.

21. The apparatus of claim 1, wherein the two or more visible beams of light intersect to delineate a treatment zone defined by an enclosed area formed by the intersection of the two or more visible beams of light.

22. The apparatus of claim 1, wherein the treatment area at the predetermined target distance is remote from the cold plasma device.

23. The apparatus of claim 1, wherein the array of light sources comprises a diode array that is electronically coordinated with a specific gas composition and tip construction used with the cold plasma device.
